(19) United States
(12) Patent Application Publication
(10) Pub. No.: US 2014/0316714 A1
(43) Pub. Date: Oct. 23, 2014

(54) METHOD FOR PREDICTING BEEF MARBLING STANDARD (BMS) NUMBERS USING COAT MINERAL COMPOSITION

(71) Applicants: School Corporation, Azabu Veterinary Medicine Educational Institution, Kanagawa (JP); Tochigi Prefecture, Tochigi (JP)

(72) Inventors: Katsuji Uetake, Sagamihara-shi (JP); Eisaku Shimada, Sagamihara-shi (JP); Hisao Nozawa, Haga-gun (JP)

(73) Assignees: School Corporation, Azabu Veterinary Medicine Educational Institution, Kanagawa (JP); Tochigi Prefexture, Tochigi (JP)

(21) Appl. No.: 14/352,520

(22) PCT Filed: Oct. 18, 2012
(86) PCT No.: PCT/JP2012/076985

§ 371 Date: Jul. 11, 2014

(30) Foreign Application Priority Data
Oct. 18, 2011 (JP) 2011-229199
Nov. 21, 2011 (JP) 2011-254343

(51) Int. Cl. G06F 19/24

(52) U.S. Cl. CPC G06F 19/24
USPC 702/19

(57) ABSTRACT

An object of the present invention is to develop a method for predicting and evaluating the meat quality during the cattle fattening stage and, more particularly, a method by which beef marbling standard (BMS) numbers which determine the price of beef carcasses on the market can be estimated during the cattle fattening stage.

The present inventors conducted intensive studies with a view to attaining the stated object. As a result, they found that particular mineral contents in cattle's hair correlate to standards for evaluation of meat quality and showed that the meat quality of cattle could be predicted and evaluated before slaughter on the basis of those mineral contents, which eventually has led to the solution of the above-described problem.

### TECHNICAL FIELD

The present invention relates to providing a method for predicting beef marbling standard (BMS) numbers by means of measuring the contents of particular mineral components in washed hairs of cattle in the later stage of fattening.

### BACKGROUND ART

The quality of meat as defined in the beef carcasses trading standards is evaluated on the basis of beef marbling standard (BMS) numbers ranging in stages between No. 1 and No. 12 (see Table 1 below). The price of any beef carcass on the market is calculated by multiplying its BMS number by its weight, so the BMS number is an important factor in the livestock industry which determines the finance thereof. Hence, livestock raisers are fattening cattle in order to gain higher BMS numbers (say, in the range of 5 to 12) on the market.

TABLE 1

| BMS number | 1 | 2 | 3 | 4 | 5 | 6 | 7 | 8 | 9 | 10 | 11 | 12 |
|---|---|---|---|---|---|---|---|---|---|---|---|---|
| Marbling standard | 0 | 0+ | 1− | 1 | 1+ | 2− | 2 | 2+ | 3− | 3 | 4 | 5 |

| | | | | | |
|---|---|---|---|---|---|
| Grade | 1 | 2 | 3 | 4 | 5 |

In today's livestock industry, BMS numbers are determined visually. Briefly, carcasses chilled for one or two days after slaughter are each cut between the sixth and seventh ribs and an expert, visually checking the inside surface of the flesh, assigns BMS numbers to the individual beef carcasses. Since BMS numbers are figures that are specified by such a technique, cattle cannot be given any definite BMS numbers as long as they are at stages of fattening before slaughter.

If livestock raisers are able to predict and evaluate the quality of meat at stages of fattening before slaughter (especially at the middle to later stage of fattening), they can get higher grades of meat quality by improving on the feeding system they are using or, alternatively, they can adjust the period of fattening and optimize the timing of shipment so that their meat is credited with the highest possible quality grade. Shipment of meat with such highest possible quality grade is also preferred for livestock raisers from the viewpoint of stabilizing farm management. One of the efforts the cattle industry is making to attain this end—predicting and evaluating the quality of meat before slaughter—are researches on techniques for predicting the quality of meat during the period of fattening. Unfortunately, however, an effective method for predicting and evaluating the meat quality obtainable at shipment by some indicatives obtained from cattle at stages of fattening before slaughter evaluated for is yet to be established in the current livestock industry.

To meet the above-described need of the livestock industry, an ultrasonic meat quality evaluating technique is being studied as a method for predicting and evaluating the quality of meat at stages of fattening, and in these several years, ultrasonic devices have been used for cattle selection at carcass contests. However, ultrasonic evaluation is known to have various problems, including the needs for not only using a very expensive apparatus but also performing an extensive operation for image capturing, and the low precision of marbling estimation which is only 52% on average. Hence, the ultrasonic evaluation technique has yet to be adopted at full scale by livestock farmers and for cattle selection.

Under these circumstances, the livestock industry still has a strong need to develop a technique by which the meat quality can be predicted and evaluated before shipment in a simpler and more precise way.

### SUMMARY OF INVENTION

### Technical Problem

An object of the present invention is to develop a method for predicting and evaluating the meat quality during the cattle fattening stage and, more particularly, a method by which beef marbling standard (BMS) numbers which determine the price beef carcasses on the market can be estimated during the cattle fattening stage.

### Solution to Problem

The present inventors conducted intensive studies with a view to attaining the stated object. As a result, the inventors round that particular mineral contents in cattle's hair correlate to standards for evaluation of meat quality and showed that the meat quality of cattle could be predicted and evaluated before slaughter on the basis of those mineral contents, which eventually has led to the solution of the above-described problem. The present inventors particularly found a correlation existing between the potassium (K) content in cattle's hair and the meat quality, or a correlation existing between the potassium (K) content as combined with the selenium (Se) content, manganese (Mn) content or iron (Fe) content, or any combination thereof and the meat quality; the inventors then showed that the meat quality of cattle could be predicted and evaluated before slaughter on the basis of those mineral contents.

Specifically, the present invention provides a method for predicting the meat quality of cattle at the middle to later stage of fattening is likely to be at the time of one to twelve months later, comprising (1) the step of measuring the potassium (K) content in the cattle's hair at the middle to later stage of fattening and (2) predicting BMS numbers of the carcasses of the cattle at one to twelve months after the measuring of the index on the basis of the measured potassium (K) content in accordance with the following equation:

BMS number=X₁+Yₖ×K content (Eq. 1).

In this Eq. 1, K content represents the measured value of potassium (K) content in cattle's hair whereas the constant term X₁ and the K concentration coefficient Yₖ are set as shown in Table 2 below, provided that X₁ and Yₖ can be specified independently of each other. By substituting the measured values of K content into this Eq. 1, BMS numbers of the carcasses of the cattle at one to twelve months after the measurement can be predicted. Note that each of the numerals in Table 2 is rounded to 4 digits after the decimal point.

TABLE 2
Correlation between K content and BMS number
BMS number = X₁ + Yₖ × K content (Eq. 1)

| | | | Residual | | Y_{K} | |
|---|---|---|---|---|---|---|
| Months to | Age in | | degree of | X₁ | (K concentration | Determination |
| shipment | months | n | freedom | (constant term) | coefficient) | coefficient |
| 1 Month | 30 | 20 | 18 | 7.8991~10.7141 | −0.003593~−0.0002865 | 0.252974 |
| 2 Months | 29 | 18 | 16 | 7.6428~10.8737 | −0.002972~0.00007216 | 0.203215 |
| 3 Months | 28 | 18 | 16 | 7.0376~10.2045 | −0.002417~0.0007373 | 0.074341 |
| 4 Months | 27 | 14 | 12 | 6.2744~10.8241 | −0.002472~0.001432 | 0.027042 |
| 5 Months | 26 | 12 | 10 | 5.7005~11.5808 | −0.003995~0.002635 | 0.02043 |
| 6 Months | 25 | 10 | 8 | 5.0917~9.2541 | −0.0008788~0.002253 | 0.113452 |
| 7 Months | 24 | 10 | 8 | 4.8878~9.9011 | −0.001663~0.002695 | 0.035917 |
| 8 Months | 23 | 10 | 8 | 4.4014~9.4400 | −0.001094~0.002872 | 0.117892 |
| 9 Months | 22 | 10 | 8 | 3.4100~11.7842 | −0.002890~0.003368 | 0.003857 |
| 10 Months | 21 | 6 | 4 | 6.0633~16.3514 | −0.007266~0.001606 | 0.439545 |
| 11 Months | 20 | 6 | 4 | 6.0273~14.3467 | −0.003957~0.001117 | 0.374737 |
| 12 Months | 19 | 6 | 4 | 1.0481~17.8484 | −0.005641~0.004081 | 0.046981 |

The present invention also provides a method for predicting when cattle at the middle to later stage of fattening should be shipped and how the meat quality of the cattle is likely to be at the time of one to twelve months later, comprising (1) the step of measuring the potassium (K) content and the selenium (Se) content, manganese (Mn) content or iron (Fe) content, or any combination thereof in the hair of the cattle at the middle to later stage of fattening and (2) predicting BMS numbers of the carcasses of the cattle at one to twelve months after the measuring in accordance with the following equation:

BMS number=X₂+Yₖ×K content+Y_{Se}×Se content+Y_{Mn}×Mn content+Y_{Fe}×Fe content (Eq. 2).

In this Eq. 2, K content represents the measured value of potassium (K) content in cattle's hair, Se content represents the measured value of selenium (Se) content in cattle's hair, Mn content represents the measured value of manganese (Mn) content in cattle's hair, and Fe content represents the measured value of iron (Fe) content in cattle's hair whereas the constant term X₂ as well as the K concentration coefficient Yₖ, the Se concentration coefficient Y_{Se}, the Mn concentration coefficient Y_{Mn}, and the Fe concentration coefficient Y_{Fe} are set as shown in Table 3 below, provided that X₂, Yₖ, Y_{Se}, Y^{Mn}, and Y_{Fe} can be specified independently of each other. By substituting the measured values of K content, Se content, Mn content, and Fe content into this Eq. 2, BMS numbers of the carcasses of the cattle at one to twelve months after the measuring can be predicted. Note that each of the numerals in Table 3 is rounded to 4 digits after the decimal point.

TABLE 3
Correlation of the contents of four elements to BMS number
BMS number = X₂ + Yₖ × K content + Y_{Se} × Se content + Y_{Mn} × Mn content + Y_{Fe} × Fe content (Eq. 2)

| | | | Residual | | Y_{K} | Y_{Se} | Y_{Mn} | Y_{Fe} | Determi- |
|---|---|---|---|---|---|---|---|---|---|
| Months to | Age in | | degree of | X₂ | (K concentration | (Se concentration | (Mn concentration | (Fe concentration | nation |
| shipment | months | n | freedom | (constant term) | coefficient) | coefficient) | coefficient) | coefficient) | coefficient |
| 1 Month | 30 | 20 | 15 | 6.4036~ | −0.003844~ | −0.001574~ | −0.4064~ | −0.06989~ | 0.320642974 |
| | | | | 11.5589 | −0.0001538 | 0.004093 | 0.3916 | 0.02048 | |
| 2 Months | 29 | 18 | 13 | 5.3839~ | −0.002849~ | −0.022845~ | −0.1290~ | −0.07258~ | 0.396613939 |
| | | | | 13.7883 | 0.0003708 | 0.007013 | 1.4436 | 0.04603 | |
| 3 Months | 28 | 18 | 13 | 1.9279~ | −0.003045~ | −0.007346~ | −0.4994~ | −0.1770~ | 0.08616972 |
| | | | | 13.9399 | 0.001040 | 0.009301 | 0.6914 | 0.1316 | |
| 4 Months | 27 | 14 | 9 | 2.8528~ | −0.002206~ | −0.003835~ | −0.7110~ | −0.4903~ | 0.289372829 |
| | | | | 10.1188 | 0.001961 | 0.004603 | 1.3385 | 0.8114 | |
| 5 Months | 26 | 12 | 7 | 4.9848~ | −0.005241~ | −0.005693~ | −0.2167~ | −0.1077~ | 0.469661876 |
| | | | | 10.7603 | 0.002222 | 0.002126 | 1.03076 | 1.002259 | |
| 6 Months | 25 | 10 | 5 | 1.6729~ | −0.0006035~ | −0.002451~ | −1.4219~ | −0.7781~ | 0.747168771 |
| | | | | 7.5312 | 0.002019 | 0.006302 | 1.1527 | 4.01645 | |
| 7 Months | 24 | 10 | 5 | 1.8994~ | −0.001940~ | −0.01260~ | −1.6538~ | −5.6811~ | 0.413979953 |
| | | | | 12.9285 | 0.003121 | 0.004211 | 3.2239 | 6.06734 | |
| 8 Months | 23 | 10 | 5 | −1.7545~ | −0.001356~ | −0.005869~ | −0.5041~ | −0.1865~ | 0.385766842 |
| | | | | 11.2591 | 0.004209 | 0.009551 | 0.4995 | 0.6722 | |
| 9 Months | 22 | 10 | 5 | 4.3076~ | −0.002427~ | −0.003721~ | −1.2492~ | −1.4584~ | 0.622441253 |
| | | | | 12.9786 | 0.003454 | 0.0006131 | 0.3134 | 3.3073 | |
| 10 Months | 21 | 6 | 1 | −95.3286~ | −0.05732~ | −0.03774~ | −21.5780~ | −38.8270~ | 0.904 |
| | | | | 124.8185 | 0.04674 | 0.04753 | 19.1416 | 38.4671 | |
| 11 Months | 20 | 6 | 1 | −65.1354~ | −0.04117~ | −0.08236~ | −8.4647~ | −42.4361~ | 0.894368591 |
| | | | | 82.8606 | 0.03457 | 0.08399 | 7.8739 | 50.3059 | |
| 12 Months | 19 | 6 | 1 | −21.3042~ | −0.003645~ | −0.01889~ | −1.02019~ | −8.1186~ | 0.997786997 |
| | | | | 18.2747 | 0.01289 | −0.0003289 | 1.1560 | 18.1591 | |

In each of Eq. 1 and Eq. 2 set out above, n represents the number of cattle subjected to the respective statistical tests and the determination coefficient, which is a numeral expressed by the square of the correlation coefficient, represents the reliability of a predicted value.

### Advantageous Effects of Invention

According to the methods of the present invention, beef marbling standard (BMS) numbers which have so far been predictable and evaluable only roughly before slaughter can be predicted before slaughter in a more convenient way, with the additional advantage that the timing of shipment can likewise be predicted. If the quality of meat or the timing of shipment can be predicted at stages of fattening before slaughter, the system of feeding cattle at the middle to later stage of fattening can be improved upon to get higher quality grades before shipment or, alternatively, the period of fattening and, hence, the timing of shipment can be so adjusted that the cattle's meat is credited with the highest possible quality grade. Shipment of cattle with such highest possible quality grade offers the additional advantage of making great contribution to stable farm management.

### DESCRIPTION OF EMBODIMENTS

The present inventors discovered that the contents of particular minerals in the hair of cattle at the later stage of fattening (in particular, the finishing stage) correlate to standards for evaluation of meat quality and exploiting this discovery, the inventors have provided methods by which the meat quality of cattle before slaughter can be predicted and evaluated on the basis of those mineral contents.

The present inventors have specifically shown that among the minerals in cattle's hair, the content of potassium (K) can be used as an index on the basis of which BMS numbers of the carcass of the cattle at one to twelve months after the measuring can be predicted.

Hence, in one embodiment of the present invention, there is provided a method for predicting the meat quality of cattle at the middle to later stage of fattening, comprising (1) the step of measuring the potassium (K) content in the cattle's hair at the middle to later stage of fattening and (2) predicting BMS numbers of the carcasses of the cattle at one to twelve months after the measuring on the basis of the measured potassium (K) content in accordance with the following equation:

BMS number=X₁+Yₖ×K content (Eq. 1).

In this Eq. 1, K content represents the measured value of potassium (K) content in cattle's hair whereas the constant term X₁ and the K concentration coefficient Yₖ are shown in Table 2 presented hereinabove.

The present inventors have also shown that cattle in the finishing stage is at the right time for shipment if the content of potassium (K) among the minerals in the cattle's hair is 450 μg/g and above and that the meat quality of the cattle at one to three months after the measuring can be predicted if there is observed a decrease in potassium (K) content, a decrease in selenium (se) content, an increase in manganese (Mn) content, a decrease in iron (Fe) content, or any combination thereof.

In another embodiment of the present invention, there is provided a method for predicting when cattle at the middle to later stage of fattening should be shipped and how the meat quality of the cattle is likely to be at the time of one to twelve months later, comprising (1) the step of measuring the potassium (K) content and the selenium (Se) content, manganese (Mn) content or iron (Fe) content, or any combination thereof in the hair of the cattle at the middle to later stage of fattening and (2) predicting BMS numbers of the carcasses of the cattle at one to twelve months after the measuring in accordance with the following equation:

BMS number=X₂+Yₖ×K content+Y_{Se}×Se content+Y_{Mn}×Mn content+Y_{Fe}×Fe content (Eq. 2).

In this Eq. 2, K content represents the measured value of potassium (K) content in cattle's hair, Se content represents the measured value of selenium (Se) content in cattle's hair, Mn content represents the measured value of manganese (Mn) content in cattle's hair, and Fe content represents the measured value of iron (Fe) content in cattle's hair whereas the constant term X₂ as well as the K concentration coefficient Yₖ, the Se concentration coefficient Y_{Se}, the Mn concentration coefficient Y_{Mn}, and the Fe concentration coefficient Y_{Fe} are shown in Table 3 presented hereinabove.

The present invention enables the meat quality of cattle—beef marbling standard (BMS) numbers—to be predicted in vivo before they are slaughtered. Since the beef carcass grade (i.e., their BMS numbers) at slaughter in the future can be predicted at the middle to later stage of fattening, cattle raising farmers are able to know the right time for shipment and can have other benefits to improve the efficiency in the cattle raising management as well as the farm management. In addition, the methods of the present invention which are adapted to measure the contents of potassium (K) and other minerals in samples such as cattle's hair feature are simple detection, so they are more useful than the conventional ultrasonic approach in terms of convenience in measurement. What is more, the methods are based on a non-invasive technique that uses cattle's hair as a sample and they have the additional advantage of being a simple procedure that permits sampling by anybody at cattle growing farms.

Livestock farmers, intending to fatten their cattle efficiently over the middle to later stage of fattening into the finishing stage, will change the composition of the feed in considerable ways and make every effort to ensure that the cattle at slaughter (shipment) will have the best possible meat quality. Take, for example, vitamin A as a nutrient in the feed; calves will be grown on a vitamin A containing feed until they are 9 to 16 months old, after which the supply of such feed is stopped but in the subsequent period from about 21 months of age to slaughter, the cattle are grown again on the feed containing vitamin A. This being the commonly adopted fattening procedure, a monitoring means that enables periodical prediction of meat quality over the middle to later stage of fattening, particularly into the finishing stage or that part of the later stage of fattening which is the closer to slaughter (shipment) is preferred because the cattle grower is able to improve on the feeding system being employed in the middle to later stage of fattening such that the quality grade of beef cattle can be elevated before shipment. Such periodical monitoring is also preferred in that the timing of shipment can be so adjusted as to ensure that the cattle will be shipped with an assignment of the highest possible BMS number.

The term “periodical” as used herein means performing inspection continuously at a specified interval which typically ranges from one week to three months, preferably from two weeks to two months, and more preferably from one to two months. By thusly performing periodical monitoring for predicting the meat quality of beef cattle at the latter part of the middle stage of their fattening into the later stage, particularly into the finishing stage, the timing of shipment can be chosen in such a way that the beef cattle at the later stage of fattening and onward will be shipped and slaughtered to yield carcasses having the highest possible BMS number. If the cattle subjected to a single monitoring for predicting their meat quality are found to have reached a stage where they are appropriate for shipment, they may be immediately shipped without performing any periodic monitoring for prediction.

The cattle to be monitored in accordance with the present invention may be of any breed as long as they are to be consumed for beef cattle and applicable breeds include not only Japanese Black Cattle but also other breeds such as crossbreed cattle and castrated Holstein fattening cattle.

Referring to the case of measuring the contents of minerals “in the hairs of cattle at the middle to later stage of fattening” according to the present invention, the cattle to be monitored is what are in the middle to later stage of fattening; in the typical case where cattle are shipped at the age of 30 months after birth, the middle stage of fattening ranges from 14 to 22 months after birth whereas the latter stage of fattening, which varies from one livestock farmer to another depending on the fattening system (feed supply system), is typically in the range of 22 to 30 months after birth; in particular, the finishing stage of fattening ranges from 28 to 30 months after birth.

The mineral components in cattle's hair that are to be measured for their contents in accordance with the present invention are potassium (K), selenium (Se), manganese (Mn), and iron (Fe). Among these, the content of potassium (K) is preferably measured from such viewpoints as the easiness of measurement and the high correlation it has with the carcass grade (i.e., BMS number) at slaughter. If the content of potassium (K) is combined with that of selenium (Se), manganese (Mn), iron (Fe), or any combination thereof, the carcass grade (i.e., BMS number) at slaughter or an appropriate timing of shipment can be predicted more accurately.

The correlations between the contents of the respective mineral components and BMS number are reviewed more specifically below. The contents of potassium (K), selenium (Se), and iron (Fe) have negative correlation with BMS number; if these minerals are contained in small enough amounts, the carcass grade (BMS number) is predicted to become satisfactory at later times (say, after one month, two months, three months, four months, six months, seven months, eight months, nine months, eleven months, or twelve months), and if the cattle are in the middle to later stage of fattening, especially at the finishing stage of fattening, the stage that has been reached by the fattening cattle can be predicted, which eventually enables predicting that they should be shipped anytime of the period ranging from one to three months later. On the other hand, the content of manganese (Mn) has positive correlation with BMS number; if this mineral is contained in large enough amounts, the carcass grade (BMS number) is predicted to become satisfactory at later times (say, after one month, two months, or three months), and it can be predicted that the cattle should be shipped anytime of the period ranging from one to three months later.

The contents of individual mineral components in the hairs of cattle at the middle to later stage of fattening are each measured as the content of the relevant mineral contained per gram of the hair but they have been found to vary with the conditions of cattle feeding. Hence, for the purposes of the present invention, mineral content measurements should preferably not be performed soon after the cattle feeding conditions are modified. Take, for example, the aforementioned case of adding vitamin A to the cattle's feed; the addition of vitamin A is continued until the cattle are 9 to 16 months old after birth and then the addition of vitamin A is stopped but in the subsequent period from about 21 to 30 months (slaughter), the addition of vitamin A is resumed. In this fattening procedure, the contents of minerals in the cattle's hair will fluctuate for a period of several months after the change in the feeding conditions, so such hair is not preferred as a sample for measurement in the present invention. It is rather preferred to carry out the predictive method of the present invention at the cattle's age of 18 to 21 months after birth (9 to 12 months before slaughter) or 26 to 30 months after birth (0 to 4 months before slaughter), where the cattle are less affected by the change in the feeding conditions.

In a certain embodiment where the concept of the present invention is applied to bovine at the later stage of fattening, in particular, the finishing stage (28 to 30 months after birth), it has been shown that the bovine's meat quality at slaughter can be predicted by BMS number based on the potassium (K) content.

Further, it has been verified that if the potassium (K) level in cattle's hair reaches 400 to 450 μg/g, the cattle will gain fat during an extended fattening period but meat of the cattle will have no “marbling” defined by possessing distribution of intramuscular fat; the above-indicated K content is therefore an index for the appropriate timing of shipment (finishing stage).

Statistical processing of the results obtained so far shows that by substituting a measured value of the potassium (K) content in a sample which is one or two months before shipment into the following equation:

BMS number=X₁+Yₖ×K content (Eq. 1)

(wherein
X₁ and Yₖ can be specified independently of each other;
K content represents the measured value of potassium (K) content in cattle's hair;
the constant term X₁ ranges from 7.6428 to 10.8737;
the K concentration coefficient Yₖ ranges from −0.003593 to −0.0002865)
the BMS number of the sample can be predicted based upon its relation with the potassium (K) content.

Alternatively, as a result of statistically processing the foregoing results obtained so far, it has been shown that predicted BMS numbers correlate with the potassium (K) content, selenium (Se) content, manganese (Mn) content or iron (Fe) content in the sample. Therefore, according to the present invention, BMS numbers can be predicted based upon the contents of two to four elements in the sample as selected from among the potassium (K) content, selenium (Se) content, manganese (Mn) content or iron (Fe) content.

By substituting the contents of four elements in a sample of cattle which is one or two months before shipment, namely, the potassium (K) content, selenium (Se) content, manganese (Mn) content and iron (Fe) content, into the following equation:

BMS number=X₂+Yₖ×K content+Y_{Se}×Se content+Y_{Mn}×Mn content+Y_{Fe}×Fe content (Eq. 2)

(wherein)
X₂, Yₖ, Y_{Se}, Y_{Mn}, and Y_{Fe} can be specified independently of each other;
K content represents a measured value of potassium (K) content in cattle's hair;
Se content represents a measured value of selenium (Se) content in cattle's hair;
Mn content represents a measured value of manganese (Mn) content in cattle's hair;
Fe content represents a measured value of iron (Fe) content in cattle's hair;
the constant term X₂ ranges from 5.3839 to 13.7883;
the K concentration coefficient Yₖ ranges from −0.003844 to 0.0003708;
the Se concentration coefficient Y_{Se} ranges from −0.022845 to 0.007013;
the Mn concentration coefficient Y_{Mn} ranges from −0.4064 to 1.4436;
the Fe concentration coefficient Y_{Fe} ranges from −0.07258 to 0.04603)
the BMS number of the sample can be predicted based upon its relation with the potassium (K), selenium (Se), manganese (Mn), and iron (Fe) contents.

The foregoing equations are relational expressions for making a rough prediction of BMS numbers based upon the content of either one element or the contents of two to a total of four elements in the sample as selected from potassium (K), selenium (Se), manganese (Mn), and iron (Fe) and the prediction does not strictly follow the indicated relations. Instead, the constant terms in the relational expressions for specific cases might fluctuate as more data are accumulated in the future.

The BMS numbers currently adopted in the livestock industry are integral values but this is not necessarily the case for the BMS numbers that are predictable the methods of the present invention and they may sometimes be expressed as decimal numbers. If values appearing after the decimal point arc obtained as predicted BMS numbers, the difference between the predicted value and the target BMS number becomes more obvious, it is expected as a side effect that the difference becomes the target for livestock farmers.

The present inventors made verifications to check for the consistency between the content of each of the mineral components described above and the beef marbling standard (BMS) number obtained after slaughter. Specifically, the correlation between the content of each of the mineral components described above and the BMS number after slaughter has been quantified. As it turned out, against the K content used as the sole index, the statistical error rate (p value) was 56.8%, 8.9% or 73.0% for the time when cattle were three months, two months or one month, respectively, before shipment (which is assumed here to be effected at the age of 30 months). Further, against the four indices used in combination. i.e., K content, Se content, Mn content and Fe content, the statistical error rate (p value) was 4.0%, 3.2% or 11.0% for the time when cattle were three months, two months or one month, respectively, before shipment. These statistical values are extremely high in terms of precision for biological prediction and, therefore, it has become possible to predict and estimate the meat quality prospectively before slaughter by making a check against the K content either independently or in combination with the Se content, Mn content, or Fe content.

Alternatively, it has been also shown that cattle at the later stage of fattening can be predicted to be at the right time for shipment if the content of potassium (K) per gram of the cattle's hair is at least 450 μg/g. It has also been shown the appropriate timing of shipment as well as the meat quality of the cattle at one to three months after the measurement can be predicted if there is observed a decrease in selenium (se) content, an increase in manganese (Mn) content, or a decrease in iron (Fe) content, or any combination thereof.

One month before shipment of cattle, the present inventors investigated the multiple correlation (multiple correlation coefficient) which the four indices of K content, Se content, Mn content and Fe content had with the BMS number after slaughter, and the p value for reliability of prediction was 11.0%; in addition, just prior to shipment (at the age of 30 months), the inventors investigated the simple correlation (correlation coefficient) which the K content measured independently had with the BMS number obtained after slaughter, and the p value for reliability of prediction w as shown to be 16.4%. In comparison between these results, it was showed that mineral contents in cattle's hair as measured three to one month before shipment (which is assumed here to be effected at the age of 30 months) correlate to the BMS number obtained after slaughter with high precision.

The contents of minerals in cattle's hair may be measured by any known methods for measurement of mineral contents, including, for example, mass spectrometer, total-reflection X-ray fluorescence spectrometer, energy dispersive X-ray fluorescence spectrometer, and an elemental analyzer (ion meter). Since the relative abundance of potassium (K) is comparatively high in cattle's hair, a portable body composition analyzer may also be used to measure its content. On the other hand, the three elements selenium (Se), manganese (Mn) and iron (Fe) are so-called “trace elements” which require a mass analyzer (ICP-MS) or other sophisticated analyzing instruments to measure, so it is more likely that these elements will be adopted as the indices at farmers assisting organizations (e.g. ZENNO and feed companies) as well as inspection agencies, etc.

The hair as a sample for measurement of mineral contents is preferably taken from the cleanest possible part of the animal's body; in the case of cattle, it is preferred to take the sample from the back or around the withers that are less likely to be stained by various kinds of dirt and the ground soil.

### EXAMPLES

### Example 1

### Measurement of Mineral Contents in Animal's Hair

In this Example, a method for measuring the concentrations of minerals in animal's hair was investigated.

1. Collecting Animal's Hair

In the first step, about one gram of sample hair was collected from each of cattle subjects. Since the methods of the present invention require trace amounts of mineral components to be measured accurately, contamination of minerals from the outside must be avoided. To this end, the sample hair was collected from body parts such as the back that were stained with minimum quantities of foreign substances, e.g. feces, mud, and ground soil. Unwanted minerals might also come from the sampling equipment, so the operator wore gloves and used fluoropolymer- or Teflon-coated scissors to perform the sampling.

2. Washing and Drying the Animal's Hair

In the next step, the contaminants adhering to the sample hair were removed. To this end, the cattle's hair was put into a centrifugal tube or the like, which was filled with pure water and subjected to ultrasonic washing or shaken either with a vortex or by hand until the foreign substances detached from the cattle's hair. In this Example, the cattle's hair was ultrasonically cleaned.

The washed cattle's hair was then dried. Any drying method may be applied as long as it can prevent minerals and other foreign substances from adhering to the cattle's hair. In this Example, the washed cattle's hair was placed on KIMTOWEL® and dried within an auto-electronic desiccator. The resultant washed and dried cattle's hair now weighing 0.1 gram was used for each run of measurement. Duplicated measurements of concentration were conducted for the same sample of cattle's hair and their average was used as a measured value for one test.

3. Acidolysis of the Cattle's Hair

To 0.1 g of sample of the washed and dried cattle's hair in a Teflon-coated vessel (MCR-6E of Milestone Inc.), 8 mL of a 60% aqueous nitric acid solution (nitric acid (1.38); Kanto Chemical Industry Co., Ltd.) was added to dilute it; the Teflon-coated vessel was sealed, mounted in a microwave heater (MLS-1200MEGA of Milestone Inc.), and then heated through stages of [250 W, 180° C.×5 min]→[400 W, 180° C.×5 min]→[500 W, 180° C.×5 min]→[venting×5 min]. After the sample cooled down sufficiently, the lysate (sample solution) was recovered.

4. Measurement with Inductively Coupled Plasma Mass Spectrometer (ICP-MS)

Using ICP-MS (ELAN6000 of PerkinElmer Inc.) in accordance with the manufacturer's instructions, the mineral contents in the sample solution were determined and those in the cattle's hair were automatically measured on the basis of the dilution ratio. ICP-MS based determination of mineral contents was performed by the internal standard method. Specifically, using potassium (K), selenium (Se), manganese (Mn) and iron (Fe) containing standard solution as a control, the concentrations of potassium (K), selenium (Se), manganese (Mn) and iron (Fe) were measured from the relative emission intensities.

### Example 2

### Measurement of Potassium (K) Content in Cattle Sample

In this Example, results arc described as were obtained by measuring the content of potassium (K) in samples of cattle's hair using the method described in Example 1.

Coats each weighing one gram were sampled from cattle subjects at the stages of twelve to one month before shipment and the concentration of potassium (K) component in each hair was measured in accordance with the method described in Example 1. The concentrations of potassium (K) in the hairs as measured in this Example are listed in Table 4 below.

TABLE 4
K concentration in cattle's hair (μg/g) and obtained values of BMS number

| Individual | Date of Sampling (in months before shipment) | | | | | | | | | | | | |
|---|---|---|---|---|---|---|---|---|---|---|---|---|---|
| No. | 12 | 11 | 10 | 9 | 8 | 7 | 6 | 5 | 4 | 3 | 2 | 1 | BMS |
| 1 | 1282 | 359 | 482 | 1367 | 760 | 315 | 495 | 1339 | 675 | 538 | 1025 | 1640 | 5 |
| 2 | 2453 | 359 | 482 | 1178 | 1237 | 1739 | 1268 | 617 | 652 | 547 | 770 | 1397 | 7 |
| 3 | 1515 | 1663 | 1500 | 342 | 1758 | 543 | 959 | 550 | 649 | 177 | 396 | 234 | 11 |
| 4 | 830 | 2541 | 1369 | 1458 | 2313 | 1535 | 3294 | 933 | 1735 | 1362 | 2006 | 493 | 9 |
| 5 | 1808 | 1772 | 1486 | 1479 | 1619 | 1259 | 995 | 861 | 897 | 359 | 405 | 554 | 9 |
| 6 | 2000 | 1870 | 1129 | 1071 | 1430 | 2094 | 1004 | 950 | 1702 | 1626 | 1186 | 608 | 8 |
| 7 | | | | 1871 | 373 | 535 | 235 | 42 | 1583 | 1011 | 1390 | 774 | 6 |
| 8 | | | | 1818 | 443 | 786 | 1110 | 917 | 1599 | 1812 | 1145 | 454 | 7 |
| 9 | | | | 1218 | 495 | 719 | 593 | 1358 | 238 | 235 | 246 | 260 | 9 |
| 10 | | | | 876 | 584 | 278 | 627 | 877 | 1609 | 867 | 429 | 336 | 8 |
| 11 | | | | | | | | 784 | 898 | 593 | 1000 | 568 | 7 |
| 12 | | | | | | | | 610 | 550 | 976 | 1684 | 794 | 5 |
| 13 | | | | | | | | | 927 | 205 | 217 | 737 | 9 |
| 14 | | | | | | | | | 1137 | 359 | 608 | 484 | 9 |
| 15 | | | | | | | | | | 1289 | 1046 | 818 | 9 |
| 16 | | | | | | | | | | 1057 | 965 | 1584 | 7 |
| 17 | | | | | | | | | | 1077 | 1087 | 672 | 6 |
| 18 | | | | | | | | | | 1546 | 1394 | 1279 | 8 |
| 19 | | | | | | | | | | | | 791 | 10 |
| 20 | | | | | | | | | | | | 512 | 8 |

Based on these results, the correlation between K content and BMS number was investigated to obtain the results shown in Table 5 below.

TABLE 5
Correlation between K content and BMS number

| Months to | Correlation | P value (two-tailed | Determination |
|---|---|---|---|
| shipment | coefficient | probability) | coefficient |
| 12 Months | −0.217 | 0.680 | 0.0470 |
| 11 Months | −0.612 | 0.196 | 0.375 |
| 10 Months | −0.663 | 0.151 | 0.440 |
| 9 Months | 0.0621 | 0.865 | 0.00386 |
| 8 Months | 0.343 | 0.331 | 0.118 |
| 7 Months | 0.190 | 0.600 | 0.0359 |
| 6 Months | 0.337 | 0.341 | 0.113 |
| 5 Months | −0.143 | 0.658 | 0.0204 |
| 4 Months | −0.164 | 0.574 | 0.0270 |
| 3 Months | −0.273 | 0.274 | 0.0743 |
| 2 Months | −0.451 | 0.0604 | 0.203 |
| 1 Month | −0.503 | 0.0238 | 0.253 |

From these results, it became clear that there is negative correlation between potassium (K) content and BMS number and that the reliability of prediction is very high at the stages of eleven to ten months before shipment and two to one month before shipment.

Based on these results, calculation was made to determine the relation between the content of potassium (K) in cattle's hair samples, say, at the stages of two to one month before shipment and the BMS number; as it turned out, BMS numbers can be expressed by the following equation:

BMS number=X₁+Yₖ×K content (Eq. 1)

[wherein
X₁ is in the range from 7.6428 to 10.8737, and
Yₖ is in the range from −0.003593 to −0.0002865].

### Example 3

### Measurement of Mineral Contents in Cattle Subjects

In this Example, an experiment was conducted to verify that in addition to potassium (K), there are other mineral components whose contents in cattle's hair correlate to the BMS number.

Specifically, hairs each weighing one gram were sampled from cattle subjects at the stages of twelve to one month before shipment and the concentrations of potassium (K), selenium (Se), manganese (Mn), and iron (Fe) in each hair were measured in accordance with the method described in Example 1. The correlations which the concentrations of potassium (K), selenium (Se), manganese (Mn), and iron (Fe) in the hairs as measured in this Example have with the BMS number are summarized in Table 6 below.

TABLE 6
Analysis of correlation which the contents of various
mineral components in cattle's hair at the stages of
twelve to one month before shipment have with BMS No.

| | | | | | Determi- |
|---|---|---|---|---|---|
| Months to | K | Mn | Fe | Se | nation |
| shipment | content | content | content | content | coefficient |
| 12 Months | 0.990 | 0.621 | 0.979 | −0.997 | 0.998 |
| | 0.0890 | 0.573 | 0.129 | 0.0483 | |
| 11 Months | −0.742 | −0.417 | 0.733 | 0.124 | 0.894 |
| | 0.468 | 0.726 | 0.476 | 0.921 | |
| 10 Months | −0.791 | −0.605 | −0.0590 | 0.825 | 0.904 |
| | 0.419 | 0.586 | 0.962 | 0.382 | |
| 9 Months | 0.197 | −0.567 | 0.407 | −0.636 | 0.622 |
| | 0.672 | 0.184 | 0.364 | 0.125 | |
| 8 Months | 0.508 | −0.00520 | 0.545 | 0.265 | 0.386 |
| | 0.245 | 0.991 | 0.206 | 0.567 | |
| 7 Months | 0.259 | 0.347 | 0.0378 | −0.498 | 0.414 |
| | 0.575 | 0.446 | 0.936 | 0.256 | |
| 6 Months | 0.527 | −0.119 | 0.613 | 0.451 | 0.747 |
| | 0.224 | 0.799 | 0.143 | 0.309 | |
| 5 Months | −0.340 | 0.504 | 0.585 | −0.378 | 0.470 |
| | 0.370 | 0.167 | 0.0983 | 0.316 | |
| 4 Months | −0.0444 | 0.225 | 0.183 | 0.0685 | 0.289 |
| | 0.897 | 0.506 | 0.590 | 0.841 | |
| 3 Months | −0.282 | 0.0961 | −0.0878 | 0.0702 | 0.0862 |
| | 0.308 | 0.733 | 0.756 | 0.804 | |
| 2 Months | −0.419 | 0.448 | −0.133 | −0.303 | 0.397 |
| | 0.120 | 0.0942 | 0.637 | 0.273 | |
| 1 Month | −0512 | −0.0102 | −0.288 | 0.238 | 0.321 |
| | 0.0356 | 0.969 | 0.262 | 0.359 | |

In each column, the top figure represents a correlation coefficient and the bottom figure represents a P value (two-tailed probability).

These results show the existence of stages with high determination coefficients at the times of twelve to nine months before shipment which are characterized by stable conditions for the addition of vitamin A to diet and the contents of potassium (K), selenium (Se), manganese (Mn), and iron (Fe) in the cattle's hairs were found to be highly correlated to the BMS number (see Table 6). It also became clear that in the finishing stage, particularly at the times of two and one month before shipment, the contents of potassium (K), selenium (Se), manganese (Mn), and iron (Fe) in the cattle's hairs correlate to the BMS number. To be more specific, the contents of potassium (K), selenium (Se), and iron (Fe) have high negative correlation to the BMS number whereas the content of manganese (Mn) has high positive correlation to the BMS number (see Table 6). It was therefore demonstrated that BMS numbers can be predicted more accurately by referring to the content of potassium (K) in combination with the contents of selenium (Se), manganese (Mn), and iron (Fe).

Based on these results, calculation was made to determine the relation the contents of potassium (K), selenium (Se), manganese (Mn) and iron (Fe) in cattle's hair samples at the stage of two months before shipment would have with respect to the BMS number at the time of, say, one or two months before shipment; as it turned out, BMS numbers are expressed by the following equation:

BMS number=X₂+Yₖ×K content+Y_{Se}×Se content+Y_{Mn}×Mn content+Y_{Fe}×Fe content (Eq. 2)

[wherein
X₂ is in the range of 5.3839 to 13.7883; and
Yₖ is in the range of −0.003844 to 0.0003708;
Y_{Se} is in the range of −0.022845 to 0.007013;
Y_{Mn} is in the range of −0.4064 to 1.4436; and
Y_{Fe} is in the range of −0.07258 to 0.04603].

The following are exemplary way s in which these results may be used with advantage: cattle waiting for shipment within several months from now are measured for the content of potassium (K) only or for the contents of potassium (K), selenium (Se), manganese (Mn) and iron (Fe) at a specified interval of, say, one month; subsequently, neat quality is predicted based solely upon the content of potassium (K) which correlates with the BMS number, assuming that shipment will be made when the content of potassium (K) having negative correlation with the BMS number has reached a guide figure of 400 to 450 μg/g. If more precise prediction of meat quality is needed, the contents of the four elements, potassium (K), selenium (Se), manganese (Mn) and iron (Fe), are used as criteria.

### INDUSTRIAL APPLICABILITY

According to the methods of the present invention, beef marbling standard (BMS) numbers which have so far been predictable and evaluatable only roughly before slaughter can be predicted before slaughter in a more convenient way, with the additional advantage that the timing of shipment can likewise be predicted. If the quality of meat or the timing of shipment can be predicted at stages of fattening before slaughter, the system of feeding cattle at the middle to later stage of fattening can be improved upon to get higher quality grades before shipment or, alternatively, the period of fattening and, hence, the timing of shipment can be so adjusted that the cattle's meat is credited with the highest possible quality grade. Shipment of cattle with such highest possible quality grade offers the additional advantage of making great contribution to a consistent operation of cattle growers.

1. A method for predicting the meat quality of cattle at the later stage of fattening, which comprises:
(1) the step of measuring the potassium (K) content in the cattle's hair at the later stage of fattening; and
(2) predicting BMS numbers of the carcasses of the cattle at one to twelve months after the measuring on the basis of the measured potassium (K) content in accordance with the following equation:
BMS number=X₁+Yₖ×K content (Eq. 1)
wherein K content represents the measured value of potassium (K) content in cattle's hair whereas the constant term X₁ and the K concentration coefficient Yₖ are shown in Table 2.

2. The method for prediction according to claim 1, wherein the meat quality of the cattle is specified to be satisfactory at one to twelve months after the measuring if the predicted BMS number is in the range of 5 to 12.

3. The method for prediction according to claim 1, which predicts how the meat quality of cattle will be at the time when they have reached the age of thirty months.

4. The method for prediction according to claim 1, wherein BMS number is predicted in accordance with the following equation:
BMS number=X₁+Yₖ×K content (Eq. 1)
wherein
X₁ and Yₖ can be specified independently of each other;
X ranges from 7.6428 to 10.8737; and
Yₖ ranges from −0.003593 to −0.0002865.

5. A method for predicting when cattle at the later stage of fattening should be shipped or how the meat quality of the cattle will be at the time of one to twelve months later, which comprises:
(1) the step of measuring the potassium (K) content and the selenium (Se) content, manganese (Mn) content or iron (Fe) content, or any combination thereof in the hair of the cattle at the later stage of fattening; and
(2) predicting BMS numbers of the carcasses of the cattle at one to twelve months after the measuring in accordance with the following equation:
BMS number=X₂+Yₖ×K content+Y_{Se}×Se content+Y_{Mn}×Mn content+Y_{Fe}×Fe content (Eq. 2)
wherein
K content represents the measured value of potassium (K) content in cattle's hair,
Se content represents the measured value of selenium (Se) content in cattle's hair,
Mn content represents the measured value of manganese (Mn) content in cattle's hair, and
Fe content represents the measured value of iron (Fe) content in cattle's hair whereas the constant term X₂ as well as the K concentration coefficient Yₖ, the Se concentration coefficient Y_{Se}, the Mn concentration coefficient Y_{Mn}, and the Fe concentration coefficient Y_{Fe} are set as shown in Table 3

6. The method for prediction according to claim 5, wherein the meat quality of the cattle is specified to be satisfactory of the carcasses of the cattle at one to three months after the measuring if the predicted BMS number is in the range of 5 to 12.

7. The method for prediction according to claim 5, which predicts how the meat quality of cattle will be at the time of two months later.

8. The method for prediction according to claim 5, wherein BMS number is predicted in accordance with the following equation:
BMS number=X₂+Yₖ×K content+Y_{Se}×Se content+Y_{Mn}×Mn content+Y_{Fe}×Fe content (Eq. 2)
wherein
X₂ is in the range of 5.3839 to 13.7883; and
Yₖ is in the range of −0.003844 to 0.0003708;
Y_{Se} is in the range of −0.022845 to 0.007013;
Y_{Mn} is in the range of −0.4064 to 1.4436; and
Y_{Fe} is in the range of −0.07258 to 0.04603.

9. The method for prediction according to claim 5, wherein steps (1) and (2) are interposed by the following step:
(A) predicting that the cattle are at the right time for shipment if the potassium (K) content is 450 μg/g or above.

10. The method for prediction according to claim 1, wherein the later stage of fattening refers to the time of twenty-nine months after birth and onward.

11. The method for prediction according to claim 1, wherein the hair is sampled from the back of the cattle.

12. The method for prediction according to claim 1, wherein the potassium (K) content in the hair is measured using a mass spectrometer, a total-reflection X-ray fluorescence spectrometer, or an elemental analyzer (ion meter).

13. The method for prediction according to claim 5, wherein the later stage of fattening refers to the time of twenty-nine months after birth and onward.

14. The method for prediction according to claim 5, wherein the hair is sampled from the back of the cattle.

15. The method for prediction according to claim 5, wherein the potassium (K) content in the hair is measured using a mass spectrometer, a total-reflection X-ray fluorescence spectrometer, or an elemental analyzer (ion meter).
